(19) United States
(12) Patent Application Publication
(10) Pub. No.: US 2025/0346619 A1
(43) Pub. Date: Nov. 13, 2025

(54) Phosphino-Quinoline-Pyridine Ligands and Methods

(71) Applicant: Chevron Phillips Chemical Company LP., The Woodlands, TX (US)

(72) Inventors: Julie A. Leseberg, Kingwood, TX (US); Michael S. Webster-Gardiner, Humble, TX (US); Brooke L. Small, Kingwood, TX (US); Daniel H. Ess, Provo, UT (US); Steven M. Bischof, Spring, TX (US)

(21) Appl. No.: 19/200,426

(22) Filed: May 6, 2025

Related U.S. Application Data
(60) Provisional application No. 63/643,596, filed on May 7, 2024.
(60) Provisional application No. 63/643,606, filed on May 7, 2024.
(60) Provisional application No. 63/643,618, filed on May 7, 2024.

(51) Int. Cl. C07F 15/02; B01J 31/12; C10G 50/00

(52) U.S. Cl. CPC C07F 15/025; B01J 31/128; C10G 50/00; B01J 2531/004; B01J 2531/842

(57) ABSTRACT

Phosphino-quinoline-pyridine ligands and compositions that include complexes, the complexes including a phosphino-quinoline-pyridine ligand and a metal coordinated to the ligand are disclosed. Methods of oligomerization, such as methods of oligomerizing alpha-olefins, performed in the presence of a complex that includes a phosphino-quinoline-pyridine ligand are also disclosed.

### CROSS-REFERENCE TO RELATED APPLICATIONS

This application is a non-provisional patent application claiming the benefit of, and priority to, U.S. Provisional Patent Application No. 63/643,606, filed May 7, 2024, U.S. Provisional Patent Application No. 63/643,596, filed May 7, 2024, U.S. Provisional Patent Application No. 63/643,618, filed May 7, 2024, each of which is incorporated by reference herein in its entirety.

### TECHNICAL FIELD

This disclosure relates to phosphino-quinoline-pyridine ligands, compositions that include the phosphino-quinoline-pyridine ligands, and methods of performing chemical reactions, such as oligomerization reactions.

### BACKGROUND

Alpha-olefin production can be achieved with a number of catalysts, such as pyridine-bisimine (PDI) catalysts and pendant donor diimine (PDD) catalysts. Each catalyst, however, typically has certain advantages and disadvantages.

PDI systems, for example, typically have higher activities and thermal stabilities, but PDD systems typically produce cleaner products and more desirable distributions. Other catalyst systems, such as those that include phosphine-iminoquinoline (PIQ) ligands, also have been used for ethylene polymerization.

There remains a need for improved ligands and catalysts for producing alpha-olefins and other products, especially ligands and catalysts that exhibit one or more advantages of both PDI and PDD catalyst systems.

### SUMMARY

Provided herein are ligands and compositions that can be used in chemical reactions, such as oligomerization reactions. The oligomerization reactions can produce relatively cleaner products having desirable distributions at improved yields.

In one aspect, ligands are provided. In some embodiments, the ligands are of formula (I):

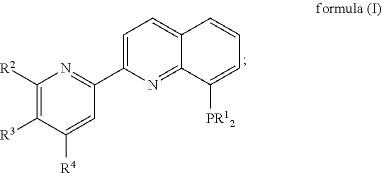

wherein R¹, R², R³, and R⁴, independently, are selected from hydrogen, a substituted or unsubstituted C₁-C₁₀ hydrocarbyl, nitro (—NO₂), sulfonate (—SO₃⁻), sulfonic acid (—SO₃H), or a halogen. In some embodiments, (a) R², (b) R³, (c) R⁴, (d) R² and R³, (e) R² and R⁴, (f) R³ and R⁴, or (g) R², R³, and R⁴ is not hydrogen.

In another aspect, compositions are provided. In some embodiments, the compositions include a complex. The complex can include a ligand, such as any of those provided herein, and a metal atom coordinated to the ligand. In some embodiments, the complexes are of formula (II):

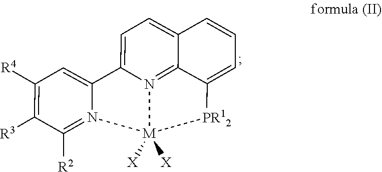

wherein R¹, R², R³, and R⁴, independently, are selected from hydrogen, a substituted or unsubstituted C₁-C₁₀ hydrocarbyl, nitro (—NO₂), sulfonate (—SO₃⁻), sulfonic acid (—SO₃H), or a halogen; wherein M is a metal atom; and wherein X is a halogen. In some embodiments, (a) R², (b) R³, (c) R⁴, (d) R² and R³, (e) R² and R⁴, (f) R³ and R⁴, or (g) R², R³, and R⁴ is not hydrogen.

In yet another aspect, methods of performing chemical reactions, such as oligomerizations, are provided. In some embodiments, the methods include providing a composition provided herein, providing an olefin, and contacting the olefin and the composition for a time and at a temperature and a pressure effective to oligomerize at least a portion of the olefin to form an oligomerized product. In some embodiments, the methods also include providing a co-catalyst, and contacting the co-catalyst and the olefin.

Additional aspects will be set forth in part in the description which follows, and in part will be obvious from the description, or may be learned by practice of the aspects described herein. The advantages described herein may be realized and attained by means of the elements and combinations particularly pointed out in the appended claims. It is to be understood that both the foregoing general description and the following detailed description are exemplary and explanatory only and are not restrictive.

### DEFINITIONS

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2^{nd} Ed (1997) can be applied, as long as that definition does not conflict with any other disclosure or definition applied herein, or render indefinite or non-enabled any claim to which that definition is applied. To the extent that any definition or usage provided by any document incorporated herein by reference conflicts with the definition or usage provided herein, the definition or usage provided herein controls.

As the various features of the subject matter of this disclosure are described, within particular aspect, a combination or combinations of the different features may be envisioned. For every aspect of every feature disclosed herein, all combinations that do not detrimentally affect the designs, compositions, systems, processes, or methods described herein are contemplated with or without the express description of that particular combination. Therefore, unless explicitly stated to the contrary, any aspect of feature disclosed here may be combined to describe and disclose the inventive designs, compositions, systems, processes, or methods consistent with the entire disclosure.

While compositions and methods are described in terms of “comprising” various components or steps, the compositions and methods can also “consist essentially of” or “consist of” the various components or steps, unless stated otherwise.

The terms “including,” “with,” and “having,” as used herein, are defined as comprising (i.e., open language), unless specified otherwise.

The terms “a,” “an,” and “the” are intended to include plural alternatives, e.g., at least one. For instance, the disclosure of “an olefin,” “a metal,” and the like, is meant to encompass one, or mixtures or combinations of more than one, olefin, metal, and the like, unless otherwise specified.

Various numerical ranges are disclosed herein. When Applicants disclose or claim a range of any type, Applicants' intent is to disclose or claim individually each possible number that such a range could reasonably encompass, including end points of the range as well as any sub-ranges and combinations of sub-ranges encompassed therein, unless otherwise specified. For example, by disclosing a concentration range of about 5 mg/m³n to about 20 mg/m³n, Applicant's intent is to recite individually 5 mg/m³n, 6 mg/m³n, 7 mg/m³n, 8 mg/m³n, 9 mg/m³n, 10 mg/m³n, 11 mg/m³n, 12 mg/m³n, 13 mg/m³n, 14 mg/m³n, 15 mg/m³n, 16 mg/m³n, 17 mg/m³n, 18 mg/m³n, 19 mg/m³n, and 20 mg/m³n, including any sub-ranges and combinations of sub-ranges encompassed therein, and these methods of describing such ranges are interchangeable. Moreover, all numerical end points of ranges disclosed herein are approximate, unless excluded by proviso. As a representative example, if Applicants state that one or more steps in the processes disclosed herein can be conducted at a temperature in a range from 10° C. to 75° C., this range should be interpreted as encompassing temperatures in a range from “about” 10° C. to “about” 75° C. unless otherwise stated.

Values or ranges may be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In another aspect, each use of the term “about” can, independently, mean ±20% of the stated value, ±15% of the stated value, ±10% of the stated value, ±5% of the stated value, or ±3% of the stated value.

Applicants reserve the right to proviso out or exclude any individual members of any such group of values or ranges, including any sub-ranges or combinations of sub-ranges within the group, that can be claimed according to a range or in any similar manner, if for any reason Applicants choose to claim less than the full measure of the disclosure, for example, to account for a reference that Applicants can be unaware of at the time of the filing of the application. Further, Applicants reserve the right to proviso out or exclude any individual substituents, analogs, compounds, ligands, structures, or groups thereof, or any members of a claimed group, if for any reason Applicants choose to claim less than the full measure of the disclosure, for example, to account for a reference or prior disclosure that Applicants can be unaware of at the time of the filing of the application.

For any particular compound or group disclosed herein, any name or structure (general or specific) presented is intended to encompass all conformational isomers, regioisomers, stereoisomers, and mixtures thereof that can arise from a particular set of substituents, unless otherwise specified. The name or structure also encompasses all enantiomers, diastereomers, and other optical isomers (if there are any) whether in enantiomeric or racemic forms, as well as mixtures of stereoisomers, as would be recognized by a skilled artisan, unless otherwise specified. For example, a general reference to a “C₆ alkyl”, “hexane” or “hexanes” includes n-hexane, 2-methylpentane, 3-methylpentane, 2,2-dimethylbutane, and 2,3-dimethylbutane; and a general reference to a “C₄ alkyl” or “butyl group” includes an n-butyl group, a sec-butyl group, an iso-butyl group, and a t-butyl group.

The term “substituted” when used to describe a group, for example, when referring to a substituted analog of a particular group, is intended to describe the compound or group wherein any non-hydrogen moiety formally replaces hydrogen in that group or compound, and is intended to be non-limiting. A compound or group can also be referred to herein as “unsubstituted” or by equivalent terms such as “non-substituted,” which refers to the original group or compound. “Substituted” is intended to be non-limiting and include inorganic substituents or organic substituents as specified and as understood by one of ordinary skill in the art.

The terms “contact product,” “contacting,” and the like, are used herein to describe compositions and methods wherein the components are contacted together in any order, in any manner, and for any length of time, unless specified otherwise. For example, the components can be contacted by blending or mixing. Further, unless otherwise specified, the contacting of any component can occur in the presence or absence of any other component of the compositions and methods described herein. Combining additional materials or components can be done by any suitable method. Further, the term “contact product” includes mixtures, blends, solutions, slurries, reaction products, and the like, or combinations thereof. Although “contact product” can, and often does, include reaction products, it is not required for the respective components to react with one another. Similarly, “contacting” two or more components can result in a reaction product or a reaction mixture. Consequently, depending upon the circumstances, a “contact product” can be a mixture, a reaction mixture, or a reaction product.

The term “alkyl group” is used herein in accordance with the definition specified by IUPAC: a univalent group formed by removing a hydrogen atom from an alkane. The alkyl group may be linear or branched unless otherwise specified.

A “cycloalkane” is used herein to refer to a saturated cyclic hydrocarbon, with or without side chains, for example, cyclobutane, cyclopentane, cyclohexane, methyl cyclopentane, and methyl cyclohexane. Other identifiers may be utilized to indicate the presence of particular groups, if any, in the cycloalkane (for example, halogenated cycloalkane indicates the presence of one or more halogen atoms replacing an equivalent number of hydrogen atoms in the cycloalkane).

The term “hydrocarbyl group” is used herein in accordance with the definition specified by IUPAC: a univalent group formed by removing a hydrogen atom from a hydrocarbon (that is, a group containing only carbon and hydrogen). Thus, a hydrocarbyl group includes alkyl groups (linear or branched), cycloalkyl groups, alkenyl groups, aryl groups, and the like. Non-limiting examples of hydrocarbyl groups include methyl, ethyl, butyl, hexyl, phenyl, tolyl, propenyl, and the like.

When used herein with regard to the selection of a substituent, the term “independently” indicates that two differently labeled substituents, e.g., R¹ and R², selected from the same pool of substituents may be the same or different.

The Abstract of this application is not intended to be used to construe the scope of the claims or to limit the scope of the subject matter that is disclosed herein, but rather to satisfy the requirements of 37 C.F.R. § 1.72(b), to enable the United States Patent and Trademark Office and the public generally to determine quickly from a cursory inspection the nature and gist of the technical disclosure. Moreover, any headings that are employed herein are also not intended to be used to construe the scope of the claims or to limit the scope of the subject matter that is disclosed herein. Any use of the past tense to describe any example otherwise indicated as constructive or prophetic is not intended to reflect that the constructive or prophetic example has actually been carried out.

All publications and patents mentioned herein are incorporated herein by reference in their entireties for the purpose of describing and disclosing, for example, the constructs and methodologies that are described in the publications, which might be used in connection with the presently described invention. The publications discussed throughout the text are provided solely for their disclosure prior to the filing date of the present application. Nothing herein is to be construed as an admission that the inventors are not entitled to antedate such disclosure by virtue of prior invention.

Those skilled in the art will readily appreciate that many modifications are possible in the exemplary embodiments disclosed herein without materially departing from the novel teachings and advantages according to this disclosure. Accordingly, all such modifications and equivalents are intended to be included within the scope of this disclosure as defined in the following claims. Therefore, it is to be understood that resort can be had to various other aspects, embodiments, modifications, and equivalents thereof which, after reading the description herein, may suggest themselves to one of ordinary skill in the art without departing from the spirit of the present disclosure or the scope of the appended claims.

### DETAILED DESCRIPTION

The present disclosure is directed to ligands, compositions, and methods of performing chemical reactions, such as oligomerizations.

### Ligands

In some embodiments, the ligands disclosed herein are phosphino-quinoline-pyridine (PQP) ligands. The ligands may include those of formula (I):

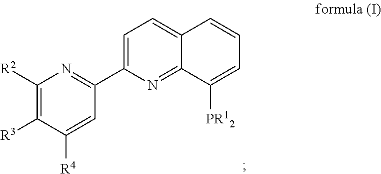

wherein R¹, R², R³, and R⁴, independently, are selected from hydrogen, a substituted or unsubstituted C₁-C₁₀ hydrocarbyl, nitro (—NO₂), sulfonate (—SO₃⁻), sulfonic acid (—SO₃H), or a halogen.

One or more of R², R³, and R⁴ of formula (I) may not be hydrogen. For example, if R² is not hydrogen, then R² can be selected from a substituted or unsubstituted C₁-C₁₀ hydrocarbyl, nitro (—NO₂), sulfonate (—SO₃⁻), sulfonic acid (—SO₃H), or a halogen. In some embodiments, R² is not hydrogen. In some embodiments, R³ is not hydrogen. In some embodiments, R⁴ is not hydrogen. In some embodiments, R² and R³ are not hydrogen. In some embodiments, R² and R⁴ are not hydrogen. In some embodiments, R³ and R⁴ are not hydrogen. In some embodiments, R², R³, and R⁴, are not hydrogen.

In formula (I), the two R¹ substituents can be the same or different. In some embodiments, the two R¹ substituents are identical. In some embodiments, the two R¹ substituents are different. Each R¹, independently, can be selected from a substituted or unsubstituted C₁-C₁₀ hydrocarbyl.

The substituted or unsubstituted C₁-C₁₀ hydrocarbyl can be a substituted or unsubstituted alkyl, such as a C₁-C₁₀ alkyl, a C₁-C₆ alkyl, a C₁-C₄ alkyl, or a C₁-C₃ alkyl. The substituted or unsubstituted C₁-C₁₀ hydrocarbyl can be straight (e.g., n-propyl) or branched (e.g., iso-propyl). The substituted or unsubstituted C₁-C₁₀ hydrocarbyl can be unsaturated (e.g., n-butyl) or saturated (e.g., (E)-but-2-enyl). The substituted or unsubstituted C₁-C₁₀ hydrocarbyl can include an aryl moiety. The substituted or unsubstituted C₁-C₁₀ hydrocarbyl can be cyclic or non-cyclic. A non-cyclic substituted or unsubstituted C₁-C₁₀ hydrocarbyl does not include any ring structures, and a cyclic substituted or unsubstituted C₁-C₁₀ hydrocarbyl includes at least one ring structure (e.g., cyclohexyl, cyclopentylmethyl, etc.). A substituted or unsubstituted C₁-C₁₀ hydrocarbyl can be substituted (e.g., mono-substituted, di-substituted, etc.) with a functional group, such as a functional group that includes an oxygen atom. The functional group including an oxygen atom can be any of those known in the art, such as a ketone, ether, alcohol, ester, etc. The substituted or unsubstituted C₁-C₁₀ hydrocarbyl can be substituted (e.g., mono-substituted, di-substituted, etc.) with a C₁-C₄ alkyl. When a C₁-C₁₀ hydrocarbyl is substituted with a substituent that includes one or more carbon atoms, the one or more carbon atoms of the substituent is included in the total count of carbon atoms; therefore, for example, a cyclohexyl substituted with an ethyl group is a C₈ hydrocarbyl.

In some embodiments, each substituted or unsubstituted C₁-C₁₀ hydrocarbyl, independently, is selected from methyl, ethyl, propyl, iso-propyl, n-butyl, iso-butyl, tert-butyl, phenyl, 3,5-dimethylphenyl, or cyclohexanone.

In some embodiments, each halogen, independently, is selected from fluoro-, chloro-, or bromo-.

In some embodiments, R², R³, and R⁴, independently, are selected from hydrogen, methyl, ethyl, iso-propyl, cyclohexanone, phenyl, nitro, sulfonate, sulfonic acid, fluoro-, chloro-, or bromo-. In some embodiments, R² is methyl or hydrogen. In some embodiments, R³, R⁴, or both R³ and R⁴ is/are hydrogen.

In some embodiments, each R¹, independently, is selected from phenyl, tert-butyl, iso-propyl, cyclohexanone, 3,5-dimethylphenyl, or iso-butyl. In some embodiments, each R¹ is phenyl, wherein, optionally, the phenyl is not substituted with one or more halogen atoms, such as a fluorine atom, at the 2-, 3-, 4-, 5-, and/or 6-position.

In some embodiments, the ligand is of formula (I), wherein each R¹ is phenyl, and each of R², R³, and R⁴ is hydrogen. The ligand of formula (I), in some embodiments, has the following structure:

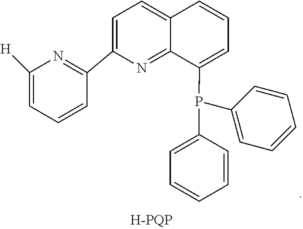 [SMILES: c1ccc(P(c2ccccc2)c2cccc3ccc(-c4ccccn4)nc23)cc1]

In some embodiments, the ligand is of formula (I), wherein each R¹ is phenyl; R² is methyl, R³ is hydrogen, and R⁴ is hydrogen. The ligand of formula (I), in some embodiments, has the following structure:

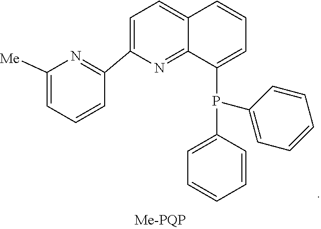 [SMILES: Cc1cccc(-c2ccc3cccc(P(c4ccccc4)c4ccccc4)c3n2)n1]

### Methods of Forming Compositions

Also provided herein are methods of forming compositions, which may include any of the ligands described herein, including the ligands of formula (I).

The methods of forming compositions can include providing a ligand, such as any of those described herein. The ligand, for example, may be H-PQP, Me-PQP, or any other ligand of formula (I).

The methods of forming compositions can include providing a metal that is capable of coordinating with a ligand, such as those of formula (I). The metal can be any that is capable of coordinating with a ligand. The metal can include iron (e.g., Fe(I), Fe(II), Fe(III), etc.), cobalt, chromium, nickel, or a combination thereof. Prior to contacting a ligand, a metal can be coordinated with an alkanoate and/or an alkanoic acid, such as octanoate and/or octanoic acid, respectively. The metal can have any oxidation number (e.g., +1, +2, +3, etc.). In some embodiments, the metal has an oxidation number of +3.

The methods of forming compositions can include contacting a ligand and a metal, wherein the contacting may be effective to coordinate an atom of the metal to the ligand. Generally, a ligand can be contacted with any amount of a metal. In some embodiments, a mole ratio of the metal to the ligand (metal: ligand) during the contacting of the ligand and the metal is about 10:1 to about 1:10, about 8:1 to about 1:8, about 6:1 to about 1:6, about 5:1 to about 1:5, about 4:1 to about 6:1, or about 5:1.

### Compositions/Catalysts

Also provided herein are compositions, which can include any ligand as described herein, such as those of formula (I), and a metal atom, which can be coordinated to the ligand. The compositions, therefore, can include a complex, wherein the complex includes (i) a ligand, such as a ligand of formula (I), and (ii) a metal atom coordinated to the ligand.

In some embodiments, the compositions include a complex of formula (II):

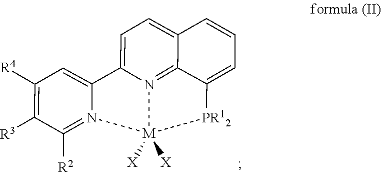

wherein R¹, R², R³, and R⁴ are as defined herein; wherein M is a metal atom; and wherein each X, independently, is a halogen. The metal atom “M” can be any metal capable of coordinating with the ligand, and, in some embodiments, the metal is iron (e.g., Fe(I), Fe(II), Fe(III), etc.), cobalt, chromium, or nickel.

The compositions provided herein can be a catalyst for a chemical reaction. Although any chemical reaction can be catalyzed by embodiments of the compositions provided herein, the chemical reaction, in some embodiments, includes an oligomerization of olefins.

The compositions provided herein generally can include one or more additional components, particularly components that do not undesirably impact the properties or effectiveness of the compositions. For example, the composition can also include a co-catalyst. When a co-catalyst is present, it can be present at any amount. In some embodiments, a co-catalyst is present in a composition at a mole ratio of co-catalyst to ligand (co-catalyst: ligand) of about 1,000:0.1 to about 1:0.1, about 1,000:0.1 to about 50:1, about 750:1 to about 50:1, about 750:1 to about 100:1, about 750:1 to about 200:1, about 750:1 to about 300:1, about 750:1 to about 400:1, about 600:1 to about 400:1, about 550:1 to about 450:1, or about 500:1 (for example, if the co-catalyst is an alkyl aluminum, the foregoing ratios can be ratios of aluminum: metal of the ligand). A co-catalyst can include any of those known in the art. In some embodiments, the co-catalyst includes an alkyl aluminum, such as tri-isobutyl aluminum (TIBA), triethylaluminum (TEA), trimethyl aluminum (TMA), methylaluminoxane (MAO), isobutyl-modified methylaluminoxane (MMAO), isobutyl-modified methylaluminoxane (TBA), or a combination thereof.

### Methods of Oligomerization

Also provided herein are methods of oligomerization.

As used herein, the terms “oligomerization”, “oligomerize”, and the like refer to processes that produce a product, which can be referred to herein as an oligomerized product, wherein the product includes oligomers and less than 1 wt % (or less than 0.1 wt %, or less than 0.01 wt %) of polymers, wherein the term “oligomers” refers to molecules that include no more than 30 carbon atoms, and the term “polymers” refers to molecules that include more than 30 carbon atoms.

In some embodiments, the methods of oligomerization include providing a composition, such as any of those provided herein, providing an olefin, and contacting the olefin and the composition. The olefin and the composition can be contacted for a time and at a temperature and a pressure effective to oligomerize at least a portion of the olefin to form an oligomerized product.

The olefin reactant can include one type of olefin, or two or more different types of olefins. The olefin reactant can include a mono-1-olefin (alpha olefin). The olefin reactant an include from 2 to 10 carbon atoms, 2 to 8 carbon atoms, or 2 to 6 carbon atoms. In some embodiments, the olefin includes ethylene, propylene, 1-butene, 1-hexene, or a combination thereof. The oligomerized product can include a C₄-C₃₀ oligomer, a C₄-C₂₆ oligomer, a C₄-C₂₂ oligomer, a C₄-C₁₈ oligomer, a C₄-C₁₆ oligomer, a C₄-C₁₂ oligomer, a C₄-C₈ oligomer, a C₆-C₃₀ oligomer, a C₈-C₃₀ oligomer, a C₁₀-C₃₀ oligomer, a C₁₂-C₃₀ oligomer, a C₁₄-C₃₀ oligomer, a C₁₆-C₃₀ oligomer, a C₁₈-C₃₀ oligomer, or a C₂₀-C₃₀ oligomer.

The oligomerization reaction can be performed in the presence of one or more other materials or components, such as a co-catalyst. For example, when a composition does not include a co-catalyst, the methods can also include providing a co-catalyst, such as any of those herein, and the contacting of the olefin and the composition can include contacting the olefin, the composition, and the co-catalyst.

The methods of oligomerization provided herein can be performed at any effective temperature. In some embodiments, the temperature is about 20° C. to about 150° C., about 20° C. to about 100° C., about 20° C. to about 80° C., about 20° C. to about 60° C., about 20° C. to about 40° C., or about 20° C. to about 30° C.

The methods of oligomerization provided herein can be provided at any effective pressure. In some embodiments, the pressure is about 10 psig to about 1,000 psig, about 10 psig to about 900 psig, about 10 psig to about 800 psig, about 10 psig to about 600 psig, about 10 psig to about 500 psig, about 100 psig to about 500 psig, about 200 psig to about 500 psig, about 300 psig to about 500 psig, about 350 psig to about 450 psig, or about 400 psig. The pressure can be imparted in any manner. In some embodiments, the pressure is imparted by an olefin (for example, the contacting may occur in a vessel, wherein a pressurized stream that includes the olefin is disposed in the vessel). In some embodiments, the contacting of the olefin and the composition occurs under a hydrogen partial pressure, wherein hydrogen can control, at least in part, oligomer molecular weight. The partial pressure of hydrogen can be from about 0 psig to about 2000 psig, about 1 psig to about 1500 psig, about 5 psig to about 1,250 psig, about 10 psig to about 1,000 psig, about 50 psig to above 750 psig, about 100 psig to about 500 psig, about 150 psig to about 400 psig, or from about 200 psig to about 300 psig.

The methods of oligomerization, particularly the contacting of an olefin and a composition, can occur for any effective time. In some embodiments, the time is about 1 minute to about 60 minutes, about 1 minute to about 50 minutes, about 1 minute to about 40 minutes, about 1 minute to about 30 minutes, about 1 minute to about 20 minutes, about 5 minutes to about 20 minutes, or about 10 minutes to about 20 minutes.

In some embodiments, an oligomerized product is produced by the methods provided herein at a yield of about 100 g/g_{cat} to about 500 g/g_{cat}, about 150 g/g_{cat} to about 400 g/g_{cat}, about 100 g/g_{cat} to about 300 g/g_{cat}, or about 150 g/g_{cat} to about 250 g/g_{cat}.

### EXAMPLES

The disclosure is further illustrated by the following examples, which are not to be construed in any way as imposing limitations to the scope of this technology. Various other aspects, embodiments, modifications, and equivalents thereof which, after reading the description herein, can suggest themselves to one of ordinary skill in the art without departing from the spirit of the present disclosure or the scope of the appended claims.

### Synthesis of Intermediates and Ligands

### Intermediate A. 8-Bromo-2-(2-pyridinyl)quinoline (PQBrH)

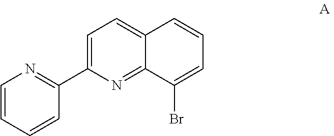 [SMILES: Brc1cccc2ccc(-c3ccccn3)nc12]

Compounds 2-amino-3-bromobenzaldehyde (0.9244 g, 4.62 mmol, 1 equiv.), 2-acetylpyridine (0.52 mL, 4.62 mmol, 1 equiv.), and KOH (0.1297 g, 2.31 mmol, 0.5 equiv.) were added to a round bottom flask in dry absolute ethanol (50 mL, dried over mol sieves) under N₂, the reaction was refluxed for 4.5 h. The reaction went from light yellow to a dark yellow/orange. The reaction was cooled to room temperature and solvents were removed in vacuo. Pure material was obtained by washing with absolute EtOH (2 mL, twice) the red filtrate was collected and put in the fridge. Filtration of the material to yield the desired product as a white solid (0.9024 g, 68% yield). ¹H NMR (400 MHz, CDCl₃), δ 8.85 (d, J=7.9, 1H), 8.74-8.72 (m, 1H), 8.66 (d, J=8.6, 1H), 8.28 (d, J=8.6, 1H), 8.07 (dd, J=7.6,1.2, 1H), 7.90 (td, J=7.7, 1.8, 1H), 7.82 (dd, J=8.12, 1.12, 1H), 7.41-7.38 (m, 2H), 1.59 (s, 3H).

### Intermediate B. 8-(diphenylphosphino)-2-(2-pyridinyl)quinoline (PQBrMe)

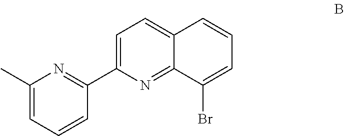 [SMILES: Cc1cccc(-c2ccc3cccc(Br)c3n2)n1]

Compounds 2-amino-3-bromobenzaldehyde (1.018 g, 5.09 mmol, 1 equiv.), 6-methyl-2-acetylpyridine (0.64 mL, 5.09 mmol, 1 equiv.), and KOH (0.1143 g, 2.04 mmol, 0.5 equiv.) were added to a round bottom flask in dry absolute ethanol (50 mL, dried over mol sieves) under N₂, the reaction was refluxed for 4.5 h. The reaction went from light yellow to a dark yellow/orange. The reaction was cooled to room temperature and solvents were removed in vacuo. Pure material was obtained by washing with absolute EtOH (2 mL, twice) then dissolving the white/yellow solid in CH₂Cl₂. The solution was filtered, the filtrate was collected, and solvents were removed in vacuo to yield the desired product (0.9229 g, 62% yield). ¹H NMR (400 MHz, CDCl₃), δ 8.70 (d, J=8.5, 1H), 8.65 (d, J=7.8, 1H), 8.25 (d, J=8.6, 1H), 8.05 (d, J=2.9, 1H), 7.79 (dd, J=5.91, 1H), 7.38 (t, J=7.78, 1H), 7.24 (d, J=7.56, 1H), 2.67 (s, 3H).

### Intermediate C: 8-(diphenylphosphino)-2-(2-pyridinyl)quinoline (PQPphenH)

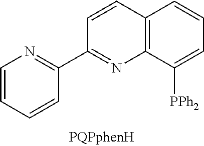

Under an inert atmosphere, a solution of A (PQBrH) (0.3179 g, 1.11 mmol, 1 equiv.) in 10 mL of dry THF was cooled to-40° C. in a round bottom flask. A solution of n-BuLi (0.73 mL, 1.05 equiv.) was added dropwise and the mixture was allowed to stir for 45 minutes, resulting in a dark red solution. Chlorodiphenylphosphine (0.21 mL, 1.28 mmol, 1.05 equiv.) was added at −40° C., the reaction solution lightened, and the mixture was allowed to warm to room temperature. The reaction was stirred overnight, and solvents were removed in vacuo. The black/brown solid was washed with 15 mL of toluene and allowed to stir for 30 minutes and white LiCl precipitate was then removed by filtration over basic alumina. Pure material was obtained by removing the solvent from the collected filtrate in vacuo. The solid was redissolved in toluene, pentane was added to the solution, and the mixture was placed in the freezer overnight to obtain an orange/yellow solid (0.1033 g, 23% yield). ¹H NMR (400 MHz, CDCl₃) δ 8.62 (dd, J=5.5, 4.0, 1H), 8.54 (d, J=8.6, 1H), 8.26 (d, J=8.6, 1H), 8.04 (d, J=8.0, 1H), 7.82 (d, J=8.1, 1H), 7.65 (td, J=7.7, 1.8, 1H), 7.44-7.39 (m, 5H), 7.34-7.32 (m, 6H), 7.25-7.23 (m, 1H), 7.12-7.09 (m, 1H); 31P NMR (202 MHz, CDCl₃), 8-12.8.

### 8-(diphenylphosphino)-2-(6-methyl-2-pyridinyl)quinoline (PQPphenMe)

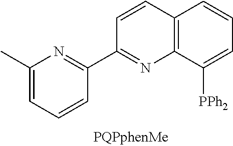

Under an inert atmosphere, a solution of B (PQBrMe) (0.3192 g, 1.06 mmol, 1 equiv.) in 10 mL of dry THF was cooled to −40° C. in a round bottom flask. A solution of n-BuLi (0.71 mL, 1.05 equiv.) was added dropwise, and the mixture was allowed to stir for 45 minutes, resulting in a dark red solution. Chlorodiphenylphosphine (0.20 mL, 1.23 mmol, 1.05 equiv.) was added at −40° C., the reaction solution lightened, and the mixture was allowed to warm to room temperature. The reaction was stirred overnight, and solvents were removed in vacuo. The yellow solid was washed with 15 mL of toluene and allowed to stir for 30 minutes, and a white LiCl precipitate was then removed by filtration over basic alumina. Pure material was obtained by removing the toluene in vacuo until ˜3 mL solvent remained. Pentane was added to the solution, and the mixture was placed in the freezer overnight to obtain an orange/yellow solid (0.110 g, 25% yield). ¹H NMR (400 MHz, CDCl₃) δ 8.60 (d, J=8.6, 1H), 8.24 (d, J=8.6, 1H), 7.86 (d, J=7.8, 1H), 7.81 (d, J=8.1, 1H), 7.54 (t, J=7.8, 1H), 7.43-7.39 (m, 5H), 7.33-7.31 (m, 6H), 7.12-7.08 (m, 2H), 2.61 (s, 3H); 31P NMR (202 MHz, CDCl₃), 8-12.6.

### Example 1

In this example, ethylene was oligomerized according to the following procedure and conditions.

Conditions/Procedure of Example 1

| Component | Amount |
|---|---|
| Ligand | 1.2 mmol |
| 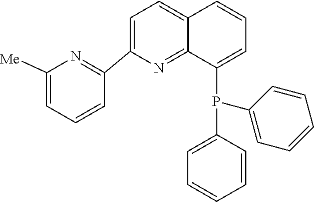 [SMILES: Cc1cccc(-c2ccc3cccc(P(c4ccccc4)c4ccccc4)c3n2)n1] | |
| Me-PQP | |
| Iron (Fe) Octanoate in C₁₂ | 5:1 (Fe:Ligand) |
| Ethylene | 400 psig (27.58 bar, gauge pressure; 2.76 |
| | MPa, gauge pressure) |
| Modified methylaluminooxane, type 3A | 500:1 (Al:Ligand) |
| (MMAO-3A) | |
| Nonane | 1.0209 g |

Reactor Temperatures of Example 1

| | |
|---|---|
| Initial Temperature | 25° C. |
| Maximum Temperature | 33° C. |
| Final Temperature | 24° C. |

A nuclear magnetic resonance (NMR) tube was filled with Fe and Me-PQP, and then capped. A charger was filled with 200 mL of cyclohexane, MMAO-3A, and an internal standard (nonane). The NMR tube was then attached to a reactor with a copper wire. The metal reactor body was then attached, and vacuum was applied to pump down the vessel. The contents of the charger were then emptied into the reactor with static vacuum. The vessel was then filled with 400 pounds per square inch (psig) (27.58 bar, gauge pressure; 2.76 MPa, gauge pressure) of ethylene. A stirrer was then turned on to break the NMR tube, which started the reaction. The reaction of this example was run for 15 minutes. The reactor was then vented, and a sample was collected. The reactants and products were analyzed using various techniques and instruments, including gas chromatography (GC).

Results of Example 1

| Product / Result | Amounts |
|---|---|
| Polymer | Trace |
| Yield (C4-C30 oligomers) | 0.0200 g |
| Productivity (g/g catalyst) | 39.98 |
| K value | 0.55 |

The foregoing results indicated a successful and efficient oligomerization of ethylene.

### Example 2

In this example, ethylene was oligomerized according to the following procedure and conditions.

Conditions/Procedure of Example 2

| Component | Amount |
|---|---|
| Ligand | 1.2 mmol |
| 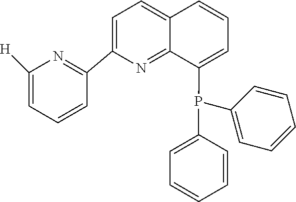 [SMILES: c1ccc(P(c2ccccc2)c2cccc3ccc(-c4ccccn4)nc23)cc1] | |
| H-PQP | |
| Iron (Fe) Octanoate in C₁₂ | 5:1 (Fe:Ligand) |
| Ethylene | 400 psig (27.58 bar, gauge pressure; 2.76 |
| | MPa, gauge pressure) |
| Modified methylaluminooxane, type 3A | 500:1 (Al:Ligand) |
| (MMAO-3A) | |
| Nonane | 1.0070 g |

Reactor Temperatures of Example 2

| | |
|---|---|
| Initial Temperature | 24° C. |
| Maximum Temperature | 33° C. |
| Final Temperature | 26° C. |

A nuclear magnetic resonance (NMR) tube was filled with Fe and H-PQP, and then capped. A charger was filled with 200 mL of cyclohexane, MMAO-3A, and an internal standard (nonane). The NMR tube was then attached to a reactor with a copper wire. The metal reactor body was then attached, and vacuum was applied to pump down the vessel. The contents of the charger were then emptied into the reactor with static vacuum. The vessel was then filled with 400 pounds per square inch (psig) (27.58 bar, gauge pressure; 2.76 MPa, gauge pressure) of ethylene. A stirrer was then turned on to break the NMR tube, which started the reaction. The reaction of this example was run for 15 minutes. The reactor was then vented, and a sample was collected. The reactants and products were analyzed using various techniques and instruments, including gas chromatography (GC).

Results of Example 2

| Product / Result | Amounts |
|---|---|
| Polymer | Trace |
| Yield (C4-C16 oligomers) | 0.1765 g |
| Productivity (g/g catalyst) | 352.98 |
| K value | 0.36 |

The foregoing results indicated a successful and efficient oligomerization of ethylene.

### ASPECTS

The following is a non-limiting listing of aspects of the disclosure:

### Ligands

Aspect 1. A ligand of formula (I):

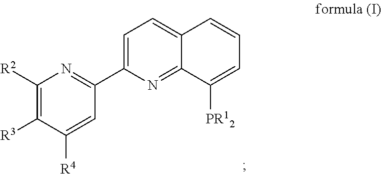

wherein R¹, R², R³, and R⁴, independently, are selected from hydrogen, a substituted or unsubstituted C₁-C₁₀ hydrocarbyl, nitro (—NO₂), sulfonate (—SO₃⁻), sulfonic acid (—SO₃H), or a halogen; and wherein, optionally, (a) R², (b) R³, (c) R⁴, (d) R² and R³, (e) R² and R⁴, (f) R³ and R⁴, or (g) R², R³, and R⁴ is not hydrogen.

Aspect 2. The ligand of Aspect 1, wherein the two R¹ substituents are identical.

Aspect 3. The ligand of Aspect 1, wherein the two R¹ substituents are different.

Aspect 4. The ligand of any of the preceding aspects, wherein each R¹, independently, is selected from a substituted or unsubstituted C₁-C₁₀ hydrocarbyl.

Aspect 5. The ligand of any of the preceding aspects, wherein the substituted or unsubstituted C₁-C₁₀ hydrocarbyl is a C₁-C₁₀ alkyl, a C₁-C₆ alkyl, a C₁-C₄ alkyl, or a C₁-C₃ alkyl.

Aspect 6. The ligand of any of the preceding aspects, wherein the substituted or unsubstituted C₁-C₁₀ hydrocarbyl is straight or branched.

Aspect 7. The ligand of any of the preceding aspects, wherein the substituted or unsubstituted C₁-C₁₀ hydrocarbyl is unsaturated or saturated.

Aspect 8. The ligand of any of the preceding aspects, wherein the substituted or unsubstituted C₁-C₁₀ hydrocarbyl comprises an aryl moiety.

Aspect 9. The ligand of any of the preceding aspects, wherein the substituted or unsubstituted C₁-C₁₀ hydrocarbyl is cyclic or non-cyclic.

Aspect 10. The ligand of any of the preceding aspects, wherein the substituted or unsubstituted C₁-C₁₀ hydrocarbyl is substituted (e.g., mono-substituted, di-substituted, etc.) with a functional group comprising an oxygen atom.

Aspect 11. The ligand of any of the preceding aspects, wherein the functional group comprising an oxygen atom is a ketone.

Aspect 12. The ligand of any of the preceding aspects, wherein the substituted or unsubstituted C₁-C₁₀ hydrocarbyl is substituted (e.g., mono-substituted, di-substituted, etc.) with a C₁-C₄ alkyl.

Aspect 13. The ligand of any of the preceding aspects, wherein each substituted or unsubstituted C₁-C₁₀ hydrocarbyl, independently, is selected from methyl, ethyl, propyl, iso-propyl, n-butyl, iso-butyl, tert-butyl, phenyl, 3,5-dimethylphenyl, or cyclohexanone.

Aspect 14. The ligand of any of the preceding aspects, wherein each halogen, independently, is selected from fluoro-, chloro-, or bromo-.

Aspect 15. The ligand of any of the preceding aspects, wherein R², R³, and R⁴, independently, are selected from hydrogen, methyl, ethyl, iso-propyl, cyclohexanone, phenyl, nitro, sulfonate, sulfonic acid, fluoro-, chloro-, or bromo-.

Aspect 16. The ligand of any of the preceding aspects, wherein each R¹, independently, is selected from phenyl, tert-butyl, iso-propyl, cyclohexanone, 3,5-dimethylphenyl, or iso-butyl.

Aspect 17. The ligand of any of the preceding aspects, wherein each R¹ is phenyl, wherein, optionally, the phenyl is not substituted with halogen atoms, such as a fluorine atom, at the 2-, 3-, 4-, 5-, and/or 6-position.

Aspect 18. The ligand of any of the preceding aspects, wherein R² is methyl or hydrogen.

Aspect 19. The ligand of any of the preceding aspects, wherein R³ is hydrogen.

Aspect 20. The ligand of any of the preceding aspects, wherein R⁴ is hydrogen.

Aspect 21. The ligand of any of the preceding aspects, wherein the ligand has the following structure:

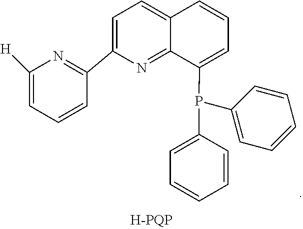 [SMILES: c1ccc(P(c2ccccc2)c2cccc3ccc(-c4ccccn4)nc23)cc1]

Aspect 22. The ligand of any of the preceding aspects, wherein the ligand has the following structure:

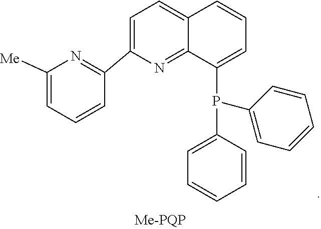 [SMILES: Cc1cccc(-c2ccc3cccc(P(c4ccccc4)c4ccccc4)c3n2)n1]

### Methods of Forming Compositions

Aspect 23. A method of forming a composition, the method comprising, consisting essentially of, or consisting of (i) providing the ligand of any of the preceding aspects, (ii) providing a metal that is capable of coordinating with any ligand of any of the preceding aspects, and (iii) contacting the ligand and the metal, wherein the contacting is effective to coordinate an atom of the metal to the ligand, wherein, optionally, the metal has an oxidation number of +3.

Aspect 24. The method of any of the preceding aspects, wherein the metal comprises, consists essentially of, or consists of iron (e.g., Fe(I), Fe(II), Fe(III), etc.), cobalt, chromium, nickel, or a combination thereof.

Aspect 25. The method of any of the preceding aspects, wherein the metal is coordinated with an alkanoate and/or an alkanoic acid prior to, during, and/or after the contacting of the metal and the ligand.

Aspect 26. The method of any of the preceding aspects, wherein the alkanoate and/or the alkanoic acid comprises, consists essentially of, or consists of octanoate and/or octanoic acid, respectively.

Aspect 27. The method of any of the preceding aspects, wherein a mole ratio of the metal to the ligand (metal: ligand) during the contacting of the ligand and the metal is about 10:1 to about 1:10, about 8:1 to about 1:8, about 6:1 to about 1:6, about 5:1 to about 1:5, about 4:1 to about 6:1, or about 5:1.

### Catalysts

Aspect 28. A composition comprising, consisting essentially of, or consisting of a complex, wherein the complex comprises, consists essentially of, or consists of (i) a ligand of any of the preceding aspects, and (ii) a metal, wherein an atom of the metal is coordinated to the ligand.

Aspect 29. A composition made according to the method of any of the preceding aspects.

Aspect 30. The composition of any of the preceding aspects, wherein the composition is a catalyst for a chemical reaction, wherein the chemical reaction is an oligomerization of olefins.

Aspect 31. The composition of any of the preceding aspects, wherein the complex comprises, consists essentially of, or consists of a complex of formula (II):

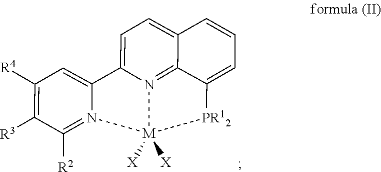

wherein R¹, R², R³, and R⁴ are as defined by any of the preceding aspects; wherein M is a metal atom; wherein each X, independently, is a halogen; and wherein, optionally, (a) R², (b) R³, (c) R⁴, (d) R² and R³, (e) R² and R⁴, (f) R³ and R⁴, or (g) R², R³, and R⁴ is not hydrogen.

Aspect 32. The composition of any of the preceding aspects, wherein M is Fe(I) or Fe(III).

Aspect 33. The composition of any of the preceding aspects, wherein the composition further comprises, consists essentially of, or consists of a co-catalyst.

Aspect 34. The composition of any of the preceding aspects, wherein the co-catalyst is present in the composition at a mole ratio of co-catalyst to ligand (co-catalyst: ligand) of about 1,000:0.1 to about 1:0.1, about 1,000:0.1 to about 50:1, about 750:1 to about 50:1, about 750:1 to about 100:1, about 750:1 to about 200:1, about 750:1 to about 300:1, about 750:1 to about 400:1, about 600:1 to about 400:1, about 550:1 to about 450:1, or about 500:1 (for example, if the co-catalyst is an alkyl aluminum, the foregoing ratios can be ratios of aluminum: metal of the ligand).

Aspect 35. The composition of any of the preceding aspects, wherein the co-catalyst comprises, consists essentially of, or consists of an alkyl aluminum.

Aspect 36. The composition of any of the preceding aspects, wherein the co-

catalyst comprises, consists essentially of, or consists of tri-isobutyl aluminum (TIBA), triethylaluminum (TEA), trimethyl aluminum (TMA), methylaluminoxane (MAO), isobutyl-modified methylaluminoxane (MMAO), isobutyl-modified methylaluminoxane (TBA), or a combination thereof.

### Methods of Oligomerization

Aspect 37. A method of oligomerization, the method comprising, consisting essentially of, or consisting of (i) providing a composition of any of the preceding aspects, (ii) providing an olefin, and (iii) contacting the olefin and the composition for a time and at a temperature and a pressure effective to oligomerize at least a portion of the olefin to form an oligomerized product.

Aspect 38. The method of any of the preceding aspects, wherein when the composition does not include a co-catalyst, the method further comprises, consists essentially of, or consists of providing a co-catalyst, such as any of those of the preceding aspects, and the contacting of the olefin and the composition further comprises, consists essentially of, or consists of contacting the olefin, the composition, and the co-catalyst.

Aspect 39. The method of any of the preceding aspects, wherein when the composition does not include a co-catalyst, the method further comprises, consists essentially of, or consists of providing a co-catalyst, such as any of those of the preceding aspects, and contacting the olefin and the co-catalyst.

Aspect 40. The method of any of the preceding aspects, wherein the temperature is about 20° C. to about 150° C., about 20° C. to about 100° C., about 20° C. to about 80° C., about 20° C. to about 60° C., about 20° C. to about 40° C., or about 20° C. to about 30° C.

Aspect 41. The method of any of the preceding aspects, wherein the pressure is about 10 psig to about 1,000 psig, about 10 psig to about 900 psig, about 10 psig to about 800 psig, about 10 psig to about 600 psig, about 10 psig to about 500 psig, about 100 psig to about 500 psig, about 200 psig to about 500 psig, about 300 psig to about 500 psig, about 350 psig to about 450 psig, or about 400 psig.

Aspect 42. The method of any of the preceding aspects, wherein the pressure is imparted by the olefin (for example, the contacting may occur in a vessel, wherein a pressurized stream comprising the olefin is disposed in the vessel).

Aspect 43. The method of any of the preceding aspects, wherein the contacting of the olefin and the composition occurs under a hydrogen partial pressure, wherein hydrogen can control, at least in part, oligomer molecular weight.

Aspect 44. The method of any of the preceding aspects, wherein the partial pressure of hydrogen is from about 0 psig to about 2000 psig, about 1 psig to about 1500 psig, about 5 psig to about 1,250 psig, about 10 psig to about 1,000 psig, about 50 psig to above 750 psig, about 100 psig to about 500 psig, about 150 psig to about 400 psig, or from about 200 psig to about 300 psig.

Aspect 45. The method of any of the preceding aspects, wherein the time is about 1 minute to about 60 minutes, about 1 minute to about 50 minutes, about 1 minute to about 40 minutes, about 1 minute to about 30 minutes, about 1 minute to about 20 minutes, about 5 minutes to about 20 minutes, or about 10 minutes to about 20 minutes.

Aspect 46. The method of any of the preceding aspects, wherein the olefin comprises, consists essentially of, or consists of two or more different types of olefins.

Aspect 47. The method of any of the preceding aspects, wherein the olefin comprises, consists essentially of, or consists of a mono-1-olefin (alpha olefin).

Aspect 48. The method of any of the preceding aspects, wherein the olefin comprises from 2 to 10 carbon atoms, 2 to 8 carbon atoms, or 2 to 6 carbon atoms.

Aspect 49. The method of any of the preceding aspects, wherein the olefin comprises, consists essentially of, or consists of ethylene, propylene, 1-butene, 1-hexene, or a combination thereof.

Aspect 50. The method of any of the preceding aspects, wherein the oligomerized product comprises, consists essentially of, or consists of a C₄-C₃₀ oligomer, a C₄-C₂₆ oligomer, a C₄-C₂₂ oligomer, a C₄-C₁₈ oligomer, a C₄-C₁₆ oligomer, a C₄-C₁₂ oligomer, a C₄-C₈ oligomer, a C₆-C₃₀ oligomer, a C₈-C₃₀ oligomer, a C₁₀-C₃₀ oligomer, a C₁₂-C₃₀ oligomer, a C₁₄-C₃₀ oligomer, a C₁₆-C₃₀ oligomer, a C₁₈-C₃₀ oligomer, or a C₂₀-C₃₀ oligomer.

Aspect 51. The method of any of the preceding aspects, wherein the oligomerized product is produced at a yield of about 100 g/g_{cat} to about 500 g/g_{cat}, about 150 g/g_{cat} to about 400 g/g_{cat}, about 100 g/g_{cat} to about 300 g/g_{cat}, or about 150 g/g_{cat} to about 250 g/g_{cat}.

What is claimed is:

1. A composition comprising a complex, wherein in the complex comprises:
(i) a ligand of formula (I): 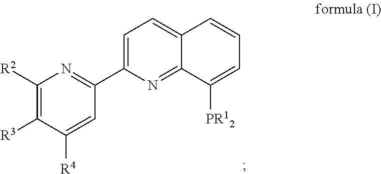
wherein R¹, R², R³, and R⁴, independently, are selected from hydrogen, a substituted or unsubstituted C₁-C₁₀ hydrocarbyl, nitro (—NO₂), sulfonate (—SO₃⁻), sulfonic acid (—SO₃H), or a halogen;
wherein (a) R², (b) R³, (c) R⁴, (d) R² and R³, (e) R² and R⁴, (f) R³ and R⁴, or (g) R², R³, and R⁴ is not hydrogen; and
(ii) a metal atom coordinated to the ligand.

2. The composition of claim 1 wherein:
(A) the metal atom is not iron, and/or
(B) (i) R², (ii) R³, (iii) R⁴, (iv) R² and R³, (v) R² and R⁴, (vi) R³ and R⁴, or (vii) R², R3, and R⁴, independently are selected from nitro (—NO₂), sulfonate (—SO₃⁻), or sulfonic acid (—SO₃H).

3. The composition of claim 1, wherein the two R¹ substituents are identical.

4. The composition of claim 1, wherein each substituted or unsubstituted C₁-C₁₀ hydrocarbyl, independently, is selected from methyl, ethyl, propyl, iso-propyl, n-butyl, iso-butyl, tert-butyl, phenyl, 3,5-dimethylphenyl, or cyclohexanone.

5. The composition of claim 1, wherein each halogen, independently, is selected from fluoro-, chloro-, or bromo-.

6. The composition of claim 1, wherein each R¹, independently, is selected from phenyl, tert-butyl, iso-propyl, cyclohexyl, 3,5-dimethylphenyl, or iso-butyl.

7. The composition of claim 1, wherein R², R³, and R⁴, independently, are selected from hydrogen, methyl, ethyl, iso-propyl, cyclohexyl, phenyl, nitro, sulfonate, sulfonic acid, fluoro-, chloro-, or bromo-.

8. The composition of claim 1, wherein each R¹ is phenyl.

9. The composition of claim 1, wherein R² is methyl or hydrogen.

10. The composition of claim 1, wherein R³ is hydrogen, and R⁴ is hydrogen.

11. The composition of claim 1, wherein the ligand has the following structure:

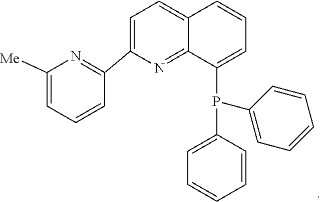 [SMILES: Cc1cccc(-c2ccc3cccc(P(c4ccccc4)c4ccccc4)c3n2)n1]

12. The composition of claim 1, wherein the complex is of formula (II): 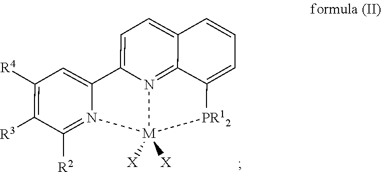
wherein R¹, R², R³, and R⁴, independently, are selected from hydrogen, a substituted or unsubstituted C₁-C₁₀ hydrocarbyl, nitro (—NO₂), sulfonate (—SO₃⁻), sulfonic acid (—SO₃H), or a halogen;
wherein M is the metal atom;
wherein X is a halogen; and
wherein (a) R², (b) R³, (c) R⁴, (d) R² and R³, (e) R² and R⁴, (f) R³ and R⁴, or (g) R², R³, and R⁴ is not hydrogen.

13. The composition of claim 12, wherein:
(A) the metal atom is not iron, and/or
(B) (i) R², (ii) R³, (iii) R⁴, (iv) R² and R³, (v) R² and R⁴, (vi) R³ and R⁴, or (vii) R², R3, and R⁴, independently is selected from nitro (—NO₂), sulfonate (—SO₃⁻), or sulfonic acid (—SO₃H).

14. The composition of claim 1, wherein the metal atom comprises iron, cobalt, chromium, nickel, or a combination thereof.

15. The composition of claim 1, wherein the composition further comprises a co-catalyst.

16. The composition of claim 15, wherein the co-catalyst comprises an alkyl aluminum.

17. The composition of claim 16, wherein the aluminum of the co-catalyst is present in the composition at a mole ratio of aluminum to the ligand of about 750:1 to about 50:1.

18. The composition of claim 16, wherein the co-catalyst comprises tri-isobutyl aluminum (TIBA), triethylaluminum (TEA), trimethyl aluminum (TMA), methylaluminoxane (MAO), isobutyl-modified methylaluminoxane (MMAO), isobutyl-modified methylaluminoxane (TBA), or a combination thereof.

19. A method of oligomerization, the method comprising:
(i) providing the composition of claim 1,
(ii) providing a co-catalyst,
(iii) providing an olefin, and
(iv) contacting the olefin, the composition, and the co-catalyst for a time and at a temperature and a pressure effective to oligomerize at least a portion of the olefin to form an oligomerized product.

20. The method of claim 19, wherein (i) the temperature is about 20° C. to about 150° C., (ii) the pressure is about 10 psig to about 1,000 psig, (iii) the time is about 1 minute to about 60 minutes, or (iv) a combination thereof.
